(19) United States
(12) Patent Application Publication
(10) Pub. No.: US 2013/0261577 A1
(43) Pub. Date: Oct. 3, 2013

(54) Ostomy belt

(71) Applicant: Andre Gene Brazeau, San Geronimo, CA (US)

(72) Inventor: Andre Gene Brazeau, San Geronimo, CA (US)

(21) Appl. No.: 13/506,167

(22) Filed: Apr. 2, 2012

(51) Int. Cl. A61F 5/449

(52) U.S. Cl. 604/345

(57) ABSTRACT

An ostomy belt with a main belt portion, a urine holding pouch portion and a stoma to pouch connector portion. The main belt portion has a semi rigid circular washer that forms an aperture toward one end of the belt. The urine holding pouch portion includes a rigid connector receiving ring, and includes a left and right hook type fastening strips attached to the inside surface of the urine pouch. The connector portion is made of low durometer silicone material and has an outwardly directed doughnut shaped ring capable of engaging a stoma in a secure leak proof and comfortable manner. An outer connector flange engages the circular washer of the belt. An inwardly facing flange engages the perimeter of the pouch connector ring. The main belt portion is wrapped around the mid section of a person and secured by the hook and loop fasteners.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of portable medical devices and more specifically to an ostomy belt system.

People who have had medical procedures for bladder cancer or other bladder related ailments sometimes result in the patient needing an appliance that directs urine out through a portion of the ureter called the stoma into a flexible pouch that is worn by the patient which retains urine that would ordinarily be exiting through the urethral opening of the patient.

To accommodate this condition, medical product manufacturers have designed and manufactured a system consisting of a belt, a urine holding pouch and a connector piece that joins the opening of the pouch to the stoma portion of the user.

However, there are deficiencies in the ostomy systems of the prior technology. First, the urine can cause the adhesive material, found on the connector flange of many common ostomy systems, to deteriorate and thereby cause leakage. Second, the standard belt that is used to hold the pouch in place is only a one inch wide elastic strap which can easily twist or roll with normal movement thereby irritating the skin tissue and further causing leaks. One manufacturer, Nuhope inc., has developed a non-adhesive system for connecting the pouch to the stoma. However, it also is known to leak and cause irritation of the skin tissue in the area under and around the connector flange. Because the connection point is so delicate, the wearer's movement is restricted, because any excessive movement causes urine leakage. This situation is very unfortunate for persons who wish to maintain an active lifestyle while coping with the unfortunate side effects of poorly designed ostomy systems.

### BRIEF SUMMARY OF THE INVENTION

The primary object of the invention is to provide an ostomy belt that reduces the amount of potential leakage between the stoma and the ostomy pouch.

Another object of the invention is to provide an ostomy belt that is secured firmly enough to the wearer to allow the wearer to have a wide range of motion during use without the danger of leakage.

Another object of the invention is to provide an ostomy belt that does not cause chafing of the wearer's skin during use.

A further object of the invention is to provide an ostomy belt that also acts to firmly retain the attached pouch during use.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed an ostomy belt comprising: a main belt portion, a urine holding pouch portion, a stoma to pouch connector portion, said main belt portion being an elongate strip having an inner facing Lycra spandex layer, a middle neoprene layer and an outer facing medical plush acetate layer, said elongate belt strip having upper and lower elongate elastomeric strips retained by sewing means to the underside of said Lycra spandex layer, said elongate belt strip having a semi rigid circular grommet forming an aperture located toward one end of said belt strip, said elongate belt strip having a hook type fastener strip sewn onto one end the outer surface of said outer medical plush acetate layer, said elongate belt strip having a loop type fastener strip sewn onto the opposite end to the outer surface of said Lycra spandex layer for purposes of attaching said belt to the mid section of a wearer, said urine holding pouch portion including a fixedly attached rigid connector receiving ring , said urine holding pouch also including a left and right hook type fastening strip each fixedly attached to the far left and right sides of the inside surface of said urine pouch capable of attaching said urine pouch to said outer layer of said belt, said connector portion being made of low durometer silicone material, said connector portion having an outwardly directed doughnut shaped ring capable of engaging a stoma in a leak proof manner, said connector portion having a concentric outwardly facing flange capable of engaging the aperture of said semi rigid circular grommet of said belt strip, said connector portion having a concentric inwardly facing flange capable of engaging the perimeter of said rigid urine pouch connector ring, said main belt portion capable of being wrapped around the mid section of a person and secured by said hook and loop fasteners, said urine pouch portion and said connector portion fastened to said main belt portion allowing said doughnut shaped ring to fit over and around the stoma of a wearer, and said urine pouch portion held to said main belt by said left and right hook and loop fastening strips and by said rigid connector receiving ring fully engaging said inwardly facing flange of said connector portion.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is an exploded view of the invention.

FIG. 2 is a side section view that bisects the invention.

FIG. 3 is a side section view of the belt of the invention.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

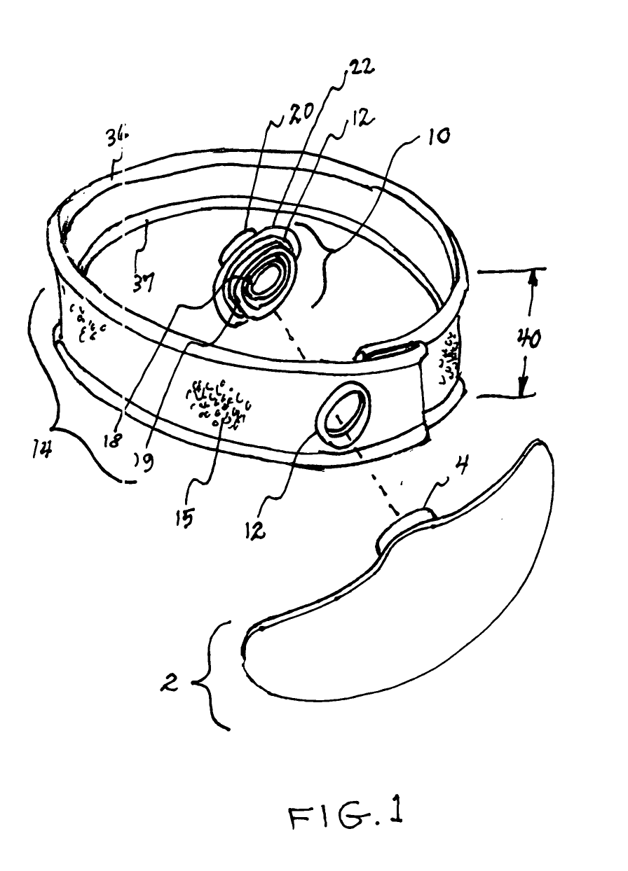

Referring now to FIG. 1 we see an exploded view of the ostomy belt system of the present invention. The invention is comprised of a belt portion 14, a connector portion 10 and a urine retaining pouch portion 2. The urine holding pouch 2 is designed in a very similar way to existing ostomy pouches on the market, except that there are no slots for holding a separate belt, and there are left and right hook type fastening strips 6, 8 fixedly attached to the inside surface of the pouch 2 that can removably attach to the loop type material 15 on the outside of the belt portion 14.

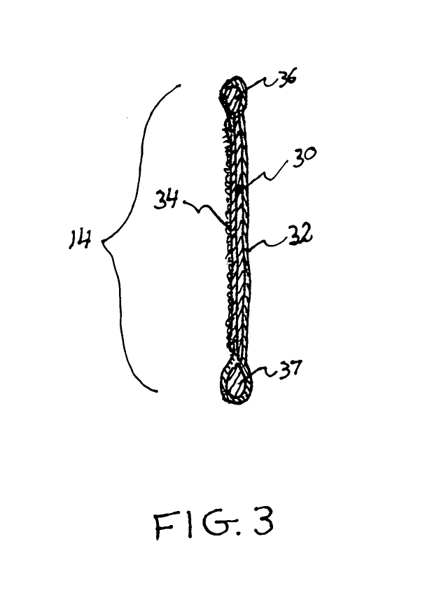

The height 10 of the belt portion 14 is approximately five and one half inches as shown by dimension arrows 40. The belt 14 assembly is made of three layers as shown in the section view in FIG. 3. The inner layer 32 is made from Lycra spandex material which is smooth and stretchable allowing for maximum comfort as it makes contact with the wearer's mid section. The middle layer 30 is made of neoprene that is approximately one sixteenth of an inch thick. The neoprene gives the belt some structure, yet is still flexible enough to be comfortable when worn. The outer layer 34 is made of medical grade plush acetate material which has the same characteristics of loop type fastening material. The belt also has an upper and lower strip 36, 37 of soft polyfoam material which are wrapped by Lycra spandex material and sewn in place. These strips 36, 37 further protect the wearer's skin from irritation and chaffing during active movement. A flexible grommet 12 is sewn into one end of the belt provides an attachment point for connector portion 10. The belt assembly 14 also acts as a back support for the wearer.

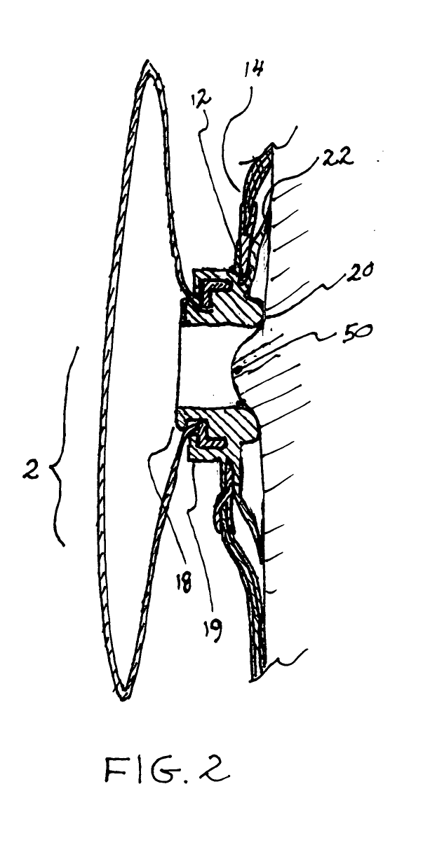

The connector portion 10 is made of medical grade low durometer silicone rubber. Referring to the section view FIG. 2, which bisects the invention, one side of the connector portion 10 is a doughnut shaped ring 20 and securely surrounds the stoma 50 of the person wearing the invention. A large secondary flange portion 22 acts as a secondary seal in case there should be any urine leakage from the doughnut shaped seal 20. A third flange portion 12 engages the flexible grommet 12 located toward one end of the belt assembly 14. An inner flange 19 surrounds and traps the inner aperture of the rigid pouch receiving ring 14. An outer flange 18 surrounds and traps the outer perimeter of rigid pouch receiving ring 14. Both the inner and outer flanges 19, 18 insure that there is no leakage between the connector 10 and the pouch 2.

The resulting novel ostomy belt assembly of the present invention is a great improvement on previous ostomy devices. The invention has been fully tested and has proven to reduce the discomfort, misery and suffering caused by previous ostomy devices, and to allow the user to maintain a relatively active lifestyle without the worry of urine leakage that can cause embarrassment and wasted time associated with the process of cleaning up and of reinstalling the device. Indeed, to test the efficacy of the present invention, the inventor, while wearing the present invention, went sky diving from a distance of twelve thousand feet and landed safely without a trace of leakage coming from the present invention. The invention can be worn from three to seven days consecutively without any signs of leaking, even when the wearer engages in robust movement.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. an ostomy belt comprising:
a main belt portion;
a urine holding pouch portion;
a stoma to pouch connector portion;
said main belt portion being an elongate strip having an inner facing Lycra spandex layer, a middle neoprene layer and an outer medical plush acetate layer;
said elongate belt strip having an upper and lower elongate elastomeric poly-foam strip retained by sewing means to the underside of said Lycra spandex layer;
said elongate belt strip having a semi rigid circular grommet forming an aperture toward one end of said belt strip;
said elongate belt strip having a hook type fastener strip sewn onto one end the outer surface of said outer medical plush acetate layer;
said elongate belt strip having a loop type fastener strip sewn onto the opposite end to the outer surface of said Lycra spandex layer;
said urine holding pouch portion including a fixedly attached rigid connector receiving ring;
said urine holding pouch also including a left and right hook type fastening strip each fixedly attached to the far left and right side of the inside surface of said urine pouch;
said connector portion being made of low durometer silicone material;
said connector portion having an outwardly directed doughnut shaped ring capable of engaging the stoma of the wearer in a leak proof manner;
said connector portion having a large, circular flange forming a secondary leak protector;
said connector portion having a concentric outwardly facing flange capable of engaging the aperture perimeter of said semi rigid circular washer of said belt strip;
said connector portion having a concentric inwardly facing flange capable of engaging the outside perimeter of said rigid urine pouch connector ring;
said main belt portion capable of being wrapped around the mid section of a person and secured by said hook and loop fasteners;
said urine pouch portion and said connector portion fastened to said main belt portion allowing said doughnut shaped ring to fit over the stoma of a wearer; and
said urine pouch portion held to said main belt by said left and right hook and loop fastening strips engaging the loop portions of said medical plush acetate belt layer and by said rigid connector receiving ring fully engaging said inwardly facing flange of said connector portion.
